(19) United States
(12) Patent Application Publication
(10) Pub. No.: US 2025/0346698 A1
(43) Pub. Date: Nov. 13, 2025

(54) POLYMER FOR THERAPEUTIC APPLICATIONS, AND PRECURSORS THEREOF

(71) Applicant: BioMimir, Inc., Sherbrooke (CA)

(72) Inventor: Elle Edin, Sherbrooke (CA)

(21) Appl. No.: 19/226,691

(22) Filed: Jun. 3, 2025

Related U.S. Application Data
(63) Continuation of application No. PCT/CA2023/051682, filed on Dec. 15, 2023.
(60) Provisional application No. 63/433,401, filed on Dec. 16, 2022.

(51) Int. Cl. C08F 230/02; A61L 24/06

(52) U.S. Cl. CPC C08F 230/02; A61L 24/06; C08F 2438/03

(57) ABSTRACT

A polymer for therapeutic purposes is provided. The polymer is an activated biocompatible polymer comprising: one or more monomer subunits of formula (iia), or one or more monomer subunits of formula (iia′):

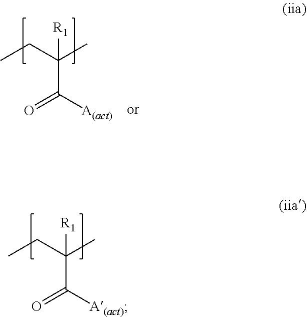

and
one or more monomer subunits of formula (ib):

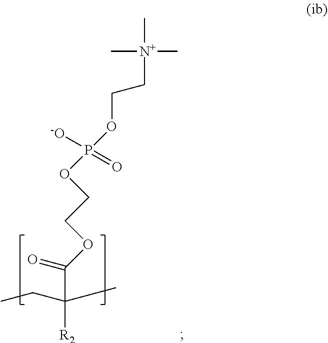

Wherein R₁ and R₂ are independently selected from H or methyl; and
A_{(act)} is an activated ester-containing group comprising at least two activated ester groups;
A′_{(act)} is an activated ester-containing group comprising at least one activated ester group;
or a salt or solvate thereof. Precursors of the activated biocompatible polymers are also provided.

### CROSS-REFERENCE TO RELATED APPLICATIONS

This application is a by-pass continuation application of PCT/CA2023/051682, filed Dec. 15, 2023, which claims the benefit of U.S. provisional patent application, 63/433,401, filed Dec. 16, 2022, the contents of each of which are incorporated by reference herein in their entirety.

### FIELD OF THE INVENTION

The present invention pertains to a polymer for therapeutic applications. More particularly, the present invention pertains to an activated biocompatible polymer for use as a medical adhesive.

### BACKGROUND

Sutures and staples have traditionally been used to close wounds due to accident or surgery. However, these conventional wound closure methods have a number of drawbacks. Sutures and staples are invasive and can cause additional damage to the tissues surrounding the wound. The use of sutures is time-consuming, and subsequent removal of sutures and staples is inconvenient and can result in additional discomfort to the wounded subject. In addition, these conventional wound closure techniques do not immediately seal the wound: thus, leakage of liquids and gases from the wounded tissue can occur. In view of the shortcomings of sutures and staples, development of alternative wound closure technologies is highly desirable. Medical polymer-based adhesives are an attractive replacement for traditional wound closure techniques, as medical polymer-based adhesives are more convenient and are easier to use, require less professional skill, and cause less trauma to the wounded subjects. As well, biomaterials that include an adhesive component can be used to bridge gaps that are too large to be pulled into contact using sutures.

Biomaterials such as collagen find use in drug delivery applications including for the treatment of burns and wounds, as well as tissue engineering. Collagen is bioresorbable/biodegradable and only weakly antigenic. As such, medical devices containing collagen are useful for wound healing and tissue engineering applications, due to the biocompatibility and safety of this biomaterial. Improved methods of incorporating such medical devices into biological systems are of significant interest.

There is a need for alternative medical polymer-based adhesives for use in therapeutic applications.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

In one aspect, there is provided a polymer comprising:
one or more monomer subunits of formula (ia):

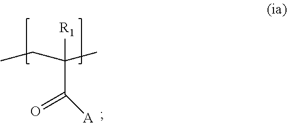

and
one or more monomer subunits of formula (ib):

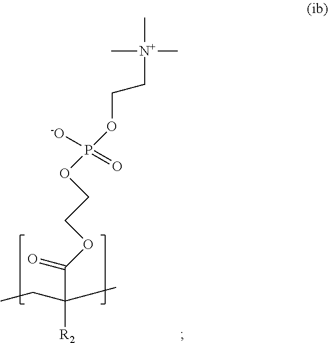

wherein
R₁ and R₂ are independently selected from H or methyl; and
A is a carboxylic acid-containing group comprising at least two —C(O)OH groups;
or a salt or solvate thereof.

In another aspect, there is provided an activated polymer comprising:
one or more monomer subunits of formula (iia):

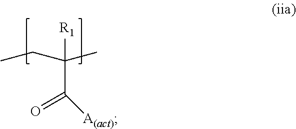

and
one or more monomer subunits of formula (ib):

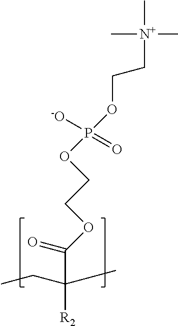

wherein
   R₁ and R₂ are independently selected from H or methyl; and
   A_{(act)} is an activated ester-containing group comprising at least two activated ester groups;
   or a salt or solvate thereof.

In yet another aspect, there is provided a polymer comprising:
one or more monomer subunits of formula (ia′):

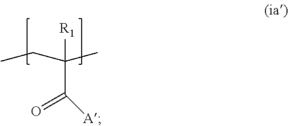

and
one or more monomer subunits of formula (ib):

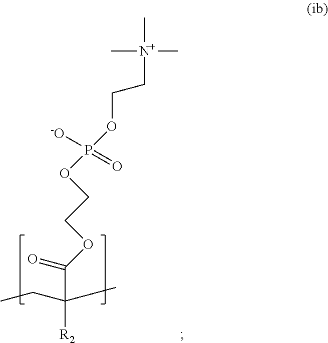

wherein
R₁ and R₂ are independently selected from H or methyl; and
A′ is a carboxylic acid-containing group comprising at least one —C(O)OH group;
or a salt or solvate thereof.

In yet another aspect, there is provided an activated polymer comprising:
one or more monomer subunits of formula (iia′):

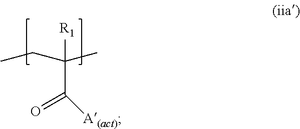

and
one or more monomer subunits of formula (ib):

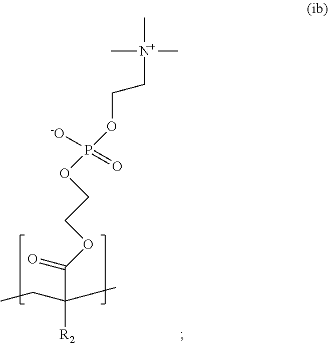

wherein
R₁ and R₂ are independently selected from H or methyl; and
A′_{(act)} is an activated ester-containing group comprising at least one activated ester group;
or a salt or solvate thereof,

### BRIEF DESCRIPTION OF THE FIGURES

The patent or application file contains at least one drawing executed in color. Copies of this patent or patent application publication with color drawing(s) will be provided by the Office upon request and payment of the necessary fee. For a better understanding of the present invention including the progression of development to get to the end product, reference is made to the following description which is to be used in conjunction with the accompanying drawings, where:

FIG. 1 is a chart illustrating percent healing of the rat surgical wounds in Example 4. Images of the rats were taken on the day of surgery, immediately pre-fill and on day 13 post-surgery. All images were analyzed using FIJI. The scale for each image was set by measuring the ruler included in each photo. The wounds were outlined using the freehand selection tool and the area was calculated using the Measure function. The percentage of total healing was calculated using the formula % H=1−(Final Area/Initial Area)*100. The samples were compared using an unpaired, one-tailed T-test with Welch's correction (p=0.0233).

FIG. 2 illustrates the histological analysis of wounds at the end of the study outlined in Example 4, showing 2× magnification H&E stained FFPE sections with inserts consisting of 20× magnified images. Scale bars represent respectively 500 and 50 μm. (A) Infiltrated lymphocytes in the granulation tissue, (B) lymphocyte aggregate in the periphery of the granulation tissue, (C) evidence of dermal edema with presence of exudate, (D) re-epithelialized wound site, (E) and granulation tissue with different lymphocytes infiltrate.

FIGS. 3a-3g illustrate various stages of a crosslinking study with the activated polymer of Example 6.

### DETAILED DESCRIPTION OF THE INVENTION

### Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) ingredient(s) and/or elements(s) as appropriate.

Terms of degree such as “substantially”, “about” and “approximately” as used herein mean a reasonable amount of deviation of the modified term such that the end result is not significantly changed. These terms of degree should be construed as including a deviation of at least ±10% of the modified term if this deviation would not negate the meaning of the word it modifies.

The term “aliphatic” refers to hydrocarbon moieties that are linear, branched or cyclic, may be alkyl, alkenyl or alkynyl (e.g. alkylene, alkenylene, or alkynyene), and may be substituted or unsubstituted.

“Substituted” means having one or more substituent moieties whose presence does not interfere with desired reactions outlined herein or the use of the polymers as described herein. In some embodiments, a substituent —C(O)OH e.g. on a precursor polymer molecule is intended to participate in subsequent reactions. In other embodiments, suitable substituents can include hydroxyl, methyl ester, or methoxy groups.

The term “biocompatible” as used herein refers to a material that is well tolerated by both the local and systemic immune system of the recipient organism. Such materials is not prone to rejection, elicits minimal inflammatory activity in the recipient organism, and causes no significant toxicity to the recipient organism.

The present application provides an activated biocompatible polymer for use as a medical adhesive, and precursor polymers to same. The activated biocompatible polymer is anti-inflammatory/anti-scarring and assists in crosslinking and in anchoring a hydrogel to a wound bed. The activated biocompatible polymer can be easily manufactured and requires minimal post synthesis processing.

The anti-inflammatory/anti-scarring property can be achieved by use of phosphorylcholine-containing monomer subunits.

The anchoring and crosslinking can be achieved using activated esters. In one embodiment, the activated esters are triazine esters. Reference to activated esters as used herein refers to esters that are prone to conjugation with primary amines. Similarly, reference to activated (biocompatible) polymers as used herein refers to polymers comprising activated ester groups.

The activated biocompatible polymer uses clusters of activated esters (e.g. triazine esters) to allow binding to both biopolymers (e.g. collagen and gelatin) as well as tissues. Specifically, the activated biocompatible polymer can bond to exposed primary amines in many proteins, including collagen, as well as in extracellular matrix. The same mechanism allows the activated biocompatible polymer to bind two different biopolymers with a single branch and act as a crosslinker. The activated biocompatible polymer can therefore create covalent bridges between adjacent tissues, or between tissues and medical devices containing collagen.

In therapeutic applications, the activated biocompatible polymer can be used to attach two surfaces containing exposed/primary amines to each other through direct application, as it both passively adheres to the surface through electrostatic interaction (van der waal and water bonds), it also binds directly with covalent bonds as the activated esters are attacked by the amines. The large size of the molecule allows for the formation of many covalent bonds on both of the surfaces to the same molecule of the activated biocompatible polymer.

In hydrogel formulations containing proteins or other organic molecules with exposed primary amines, the activated biocompatible polymer can both act as an anchor to a surface by the mechanism described above, and it also acts as a cross-linking agent as single activated biocompatible polymer molecules bind to several of the proteins (or other organic molecules).

The activated biocompatible polymer is highly hydrophilic and will stay hydrated in ambient humidity.

The properties mentioned above means that the activated biocompatible polymer fills several important functions as a bonding agent, cross-linking agent, and as a glue.

In one embodiment, there is provided a polymer comprising:
one or more monomer subunits of formula (ia):

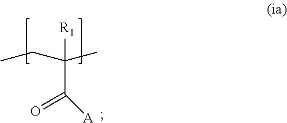

and
one or more monomer subunits of formula (ib):

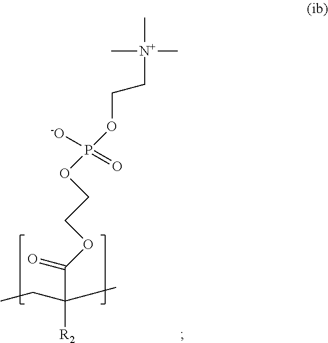

wherein
R₁ and R₂ are independently selected from H or methyl; and
A is a carboxylic acid-containing group comprising at least two —C(O)OH groups;
or a salt or solvate thereof.

The above-noted polymer is a precursor to activated polymers described in further detail below.

In one embodiment, the polymer has a number average molecular weight (Mn) of from about 2.5 kDa to about 3.6 MDa, or of from about 90 kDa to about 3.6 MDa.

In one embodiment, the salt is a physiologically acceptable salt, such as a hydrochloride salt. In another embodiment, the solvate is a physiologically acceptable solvate, such as a water or ethanol solvate. Physiologically acceptable salts and solvates will be known to the skilled worker.

In one embodiment, R₂ is methyl. In another embodiment, R₁ is H.

In another embodiment, the monomer subunits of formula (ia) and the monomer subunits of formula (ib) are present in a ratio of from about 4:1 to about 1:15.

In yet another embodiment, A is selected from formula (A-1) or formula (A-2):

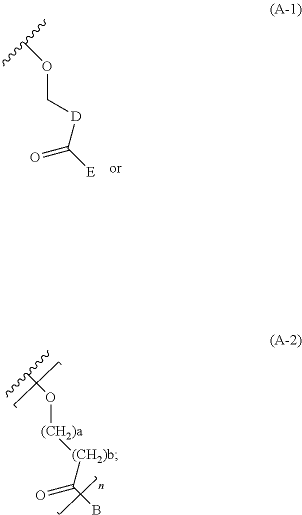

wherein D is a substituted C₃ to C₉ aliphatic group that is linear, branched or cyclic, optionally wherein one or more C of the aliphatic group is replaced by —O—, —NH—, —O—C(O)—, or —C(O)—O—;
wherein the aliphatic group comprises at least one substituent —C(O)E;
wherein each E is independently selected from —OH or B;
wherein each B is independently selected from:

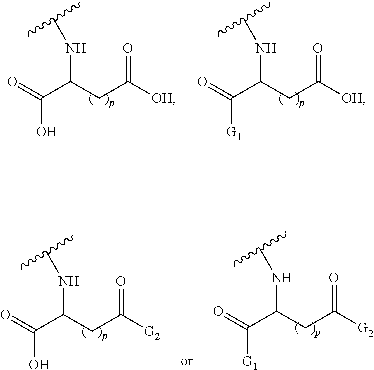

wherein each of G₁ and G₂ is independently selected from:
(a) an amino acid residue of the formula G₃:

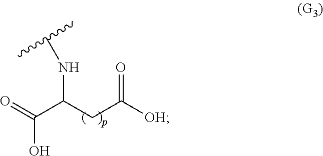

or
(b) a poly(amino acid) group formed from a condensation reaction of G₃ and one or more amino acids of the formula:

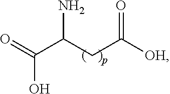

wherein the poly(amino acid) group comprises one or more peptide bonds and/or one or more isopeptide bonds;
wherein n is an integer from 1 to 3;
wherein each a and b is independently an integer from 1 to 2; and
wherein each p is independently an integer from 1 to 2.

The aliphatic group can comprise further substituents whose presence does not interfere with the polymerization reaction, or the preparation and use of activated polymers prepared from this polymer as outlined below. For example, one or more C of the aliphatic group can comprise at least one substituent selected from hydroxyl, methyl ester, or methoxy; and/or, one or more C of the aliphatic group can be —C(O)—.

In yet another embodiment, D is a substituted C₃ to C₉ alkylene group that is linear, branched or cyclic. In another embodiment, D is —(CR₄R₅)—(CR₄R₅)—(CR₄R₅)—, wherein each R₄ and each R₅ is independently selected from —H and —C(O)OH.

In still yet another embodiment, A is selected from:

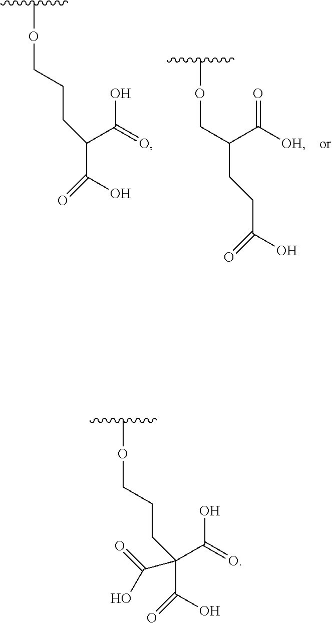 [SMILES: *OCCCC(C(=O)O)C(=O)O ; *OCC(CCC(=O)O)C(=O)O ; *OCCCC(C(=O)O)(C(=O)O)C(=O)O]

In still yet another embodiment A is selected from:

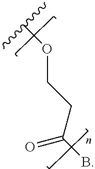

In another aspect, there is provided an activated polymer comprising:
one or more monomer subunits of formula (iia):

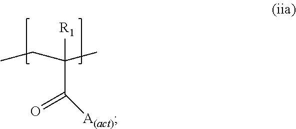

and
one or more monomer subunits of formula (ib):

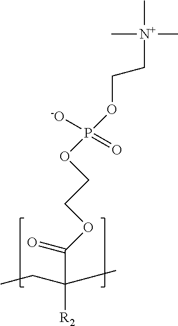

wherein
   R₁ and R₂ are independently selected from H or methyl; and
   A_{(act)} is an activated ester-containing group comprising at least two activated ester groups;
   or a salt or solvate thereof.

In one embodiment, the salt is a physiologically acceptable salt, such as a hydrochloride salt. In another embodiment, the solvate is a physiologically acceptable solvate, such as a water or ethanol solvate. Physiologically acceptable salts and solvates will be known to the skilled worker.

In one embodiment, R₂ is methyl. In another embodiment, R₁ is H.

In another embodiment, the monomer subunits of formula (ia) and the monomer subunits of formula (ib) are present in a ratio of from about 4:1 to about 1:15.

In another embodiment, A_{(act)} is selected from formula (A_{(act)}−1) or formula (A_{(act)}−2):

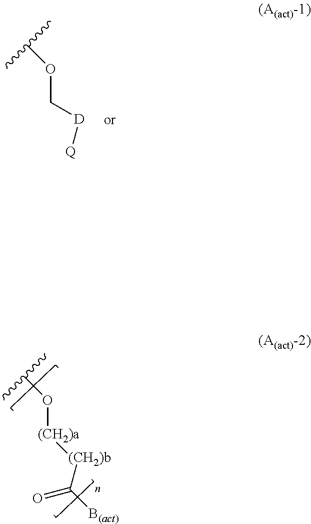

wherein D is a substituted Ca to Co aliphatic group that is linear, branched or cyclic, optionally wherein one or more C of the aliphatic group is replaced by —O—, —NH—, —O—C(O)—, or —C(O)—O—;
wherein the aliphatic group comprises at least one substituent Q;
wherein each B_{(act)} is independently selected from:

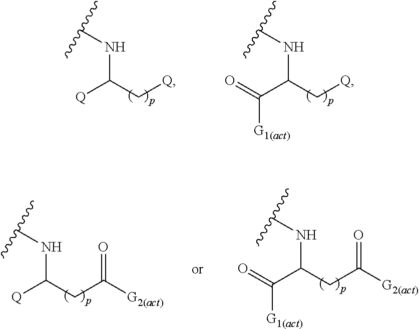

wherein each Q is an activated ester group;
wherein each of G_{1(act)} and G_{2(act)} is independently selected from:
(a) an amino acid residue of the formula G_{3(act)} comprising two activated ester groups Q:

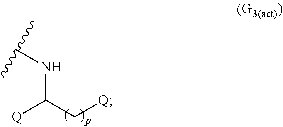

or
(b) a poly(amino acid) group formed from a condensation reaction of an amino acid residue of the formula G₃:

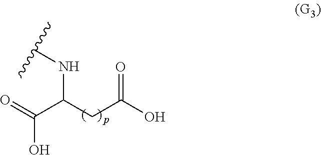

and one or more amino acids of the formula:

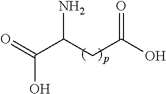

wherein the poly(amino acid) group comprises one or more peptide bonds and/or one or more isopeptide bonds, and wherein the poly(amino acid) group comprises at least two activated ester groups Q;
wherein each activated ester group Q corresponds to one of the at least two activated ester groups of A_{(act)};
wherein n is an integer from 1 to 3;
wherein each a and b is independently an integer from 1 to 2; and
wherein each p is independently an integer from 1 to 2.

The aliphatic group can comprise further substituents. For example, one or more C of the aliphatic group can comprise at least one substituent selected from hydroxyl, methyl ester, or methoxy; and/or, one or more C of the aliphatic group can be —C(O)—.

In another embodiment, D is a substituted C₃ to C₉ alkylene group that is linear, branched or cyclic. In another embodiment, D is —(CR₄R₅)—(CR₄R₅)—(CR₄R₅)—, wherein each R₄ and each R₅ is independently selected from —H and -Q.

In yet another embodiment, A_{(act)}; is selected from:

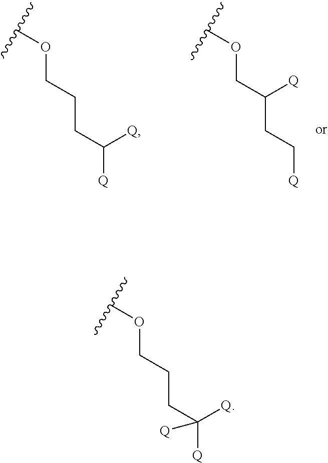

In another embodiment, A_{(act)} is selected from:

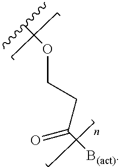

In another embodiment, each of the at least two activated ester groups comprises an N-hydroxysuccinimide ester, such as O—(N-hydroxysucccinimide) ester or O—(N-hydroxysulfosuccinimide) ester; or, a triazine ester, such as an O-(4,6-dimethoxyl-1,3,5-triazin-2-yl) ester. In another embodiment, each of the at least two activated ester groups comprises an O-(4,6-dimethoxyl-1,3,5-triazin-2-yl) ester.

In another embodiment, the activated polymer has a number average molecular weight (Mₙ) of from about 5.9 kDa to about 4 MDa, or from about 100 kDa to about 4 MDa. In one embodiment, the activated polymer is derived from a polymer precursor prepared using free radical polymerization (FRP) and has a number average molecular weight (Mₙ) of from about 400 kDa to about 4 MDa. In another embodiment, the activated polymer is derived from a polymer precursor prepared using RAFT controlled radical polymerization and has a number average molecular weight (Mₙ) of from about 5.9 kDa to about 400 kDa.

In yet another embodiment, the activated polymer is biocompatible.

In another embodiment, there is provided an activated biocompatible polymer for use as a medical adhesive, wherein the activated biocompatible polymer is prepared by a process comprising:
obtaining the polymer derived from the one or more monomer subunits of formula (ia) and one or more monomer subunits of formula (ib) as described herein, or the salt or solvate thereof;
converting the at least two —C(O)OH groups to activated ester groups; and
optionally, isolating the activated biocompatible polymer or a physiologically acceptable salt or solvate thereof.

In another embodiment, each of the activated ester groups comprises an N-hydroxysuccinimide ester, such as O—(N-hydroxysucccinimide) ester or O—(N-hydroxysulfosuccinimide) ester; or, a triazine ester, such as an O-(4,6-dimethoxyl-1,3,5-triazin-2-yl) ester. In another embodiment, each of the at least two activated ester groups comprises an O-(4,6-dimethoxyl-1,3,5-triazin-2-yl) ester.

In yet another embodiment, the activated biocompatible polymer has a number average molecular weight (Mₙ) of from about 5.9 kDa to about 4 MDa, or from about 100 kDa to about 4 MDa.

In another embodiment, there is provided an activated polymer comprising:
one or more monomer subunits of formula (iia′):

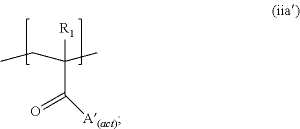

and
one or more monomer subunits of formula (ib

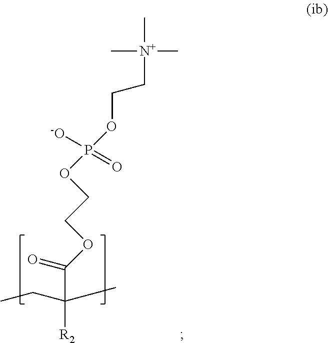

wherein
R₁ and R₂ are independently selected from H or methyl; and
A′_{(act)} is an activated ester-containing group comprising at least one activated ester group;
or a salt or solvate thereof.

In one embodiment, the salt is a physiologically acceptable salt, such as a hydrochloride salt. In another embodiment, the solvate is a physiologically acceptable solvate, such as a water or ethanol solvate. Physiologically acceptable salts and solvates will be known to the skilled worker.

In another embodiment, R₂ is methyl. In another embodiment, R₁ is H.

In another embodiment, the monomer subunits of formula (iia′) and the monomer subunits of formula (ib) are present in a ratio of from about 4:1 to about 1:15.

In still another embodiment, A′_{(act)} is selected from formula (A′_{(act)}−1) or formula (AA′_{(act)}−2):

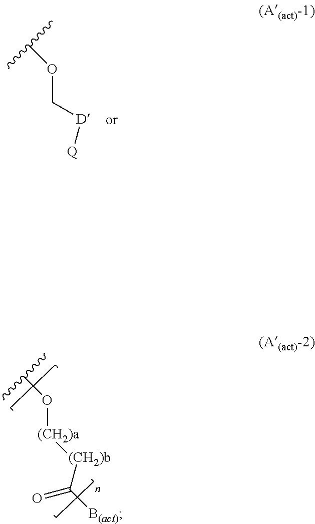

wherein D′ is an optionally substituted C₃ to C₉ aliphatic group that is linear, branched or cyclic, optionally wherein one or more C of the aliphatic group is replaced by —O—, —NH—, —O—C(O)—, or —C(O)O—;
wherein the aliphatic group optionally comprises at least one substituent Q;
wherein each Q is an activated ester group;
wherein each activated ester group Q corresponds to one of the at least one activated ester groups of group A′_{(act)};
wherein n is an integer from 1 to 3; and
wherein each a and b is independently an integer from 1 to 2.

The aliphatic group can comprise further substituents—for example, one or more C of the aliphatic group can comprise at least one substituent selected from hydroxyl, methyl ester, or methoxy; and/or, one or more C of the aliphatic group can be —C(O)—.

In yet another embodiment, D′ is an optionally substituted C₃ to C₉ alkylene group that is linear, branched or cyclic. In another embodiment, D′ is —(CR₄R₅)—(CR₄R₅)—(CR₄R₅)—, wherein each R₄ and each R₅ is independently selected from —H and -Q.

In still another embodiment, A′_{(act)} is selected from:

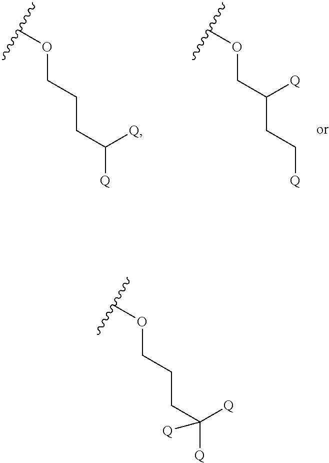

In another embodiment, A′_{(act)} is selected from:

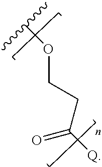

In another embodiment, each of the at least one activated ester groups comprises an N-hydroxysuccinimide ester, such as O—(N-hydroxysucccinimide) ester or O—(N-hydroxysulfosuccinimide) ester; or, a triazine ester, such as an O-(4,6-dimethoxyl-1,3,5-triazin-2-yl) ester. In another embodiment, each of the at least one activated ester groups comprises an O-(4,6-dimethoxyl-1,3,5-triazin-2-yl) ester.

In still another embodiment, the activated polymer has a number average molecular weight (Mₙ) of from about 5.9 kDa to about 4 MDa.

In another embodiment, the activated polymer is biocompatible.

In yet another embodiment, there is provided a polymer comprising:
one or more monomer subunits of formula (ia′):

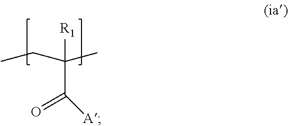

and
one or more monomer subunits of formula (ib):

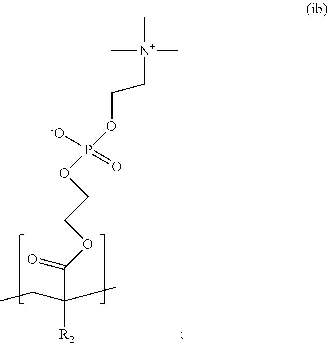

wherein
R₁ and R₂ are independently selected from H or methyl; and
A′ is a carboxylic acid-containing group comprising at least one —C(O)OH group;
or a salt or solvate thereof.

The above-noted polymer is a precursor to activated polymers comprising monomer subunit (iia′) described in further detail above.

In one embodiment, the polymer has a number average molecular weight (Mn) of from about 2.5 kDa to about 3.6 MDa, or of from about 90 kDa to about 3.6 MDa.

In one embodiment, the salt is a physiologically acceptable salt, such as a hydrochloride salt. In another embodiment, the solvate is a physiologically acceptable solvate, such as a water or ethanol solvate. Physiologically acceptable salts and solvates will be known to the skilled worker.

In another embodiment, R₂ is methyl. In another embodiment, R₁ is H.

In still another embodiment, the monomer subunits of formula (ia′) and the monomer subunits of formula (ib) are present in a ratio of from about 4:1 to about 1:15.

In yet another embodiment, A′ is selected from formula (A′-A1) or formula (A′-2):

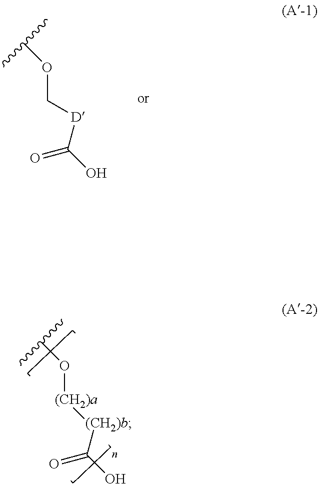

wherein D′ is an optionally substituted C₃ to C₉ aliphatic group that is linear, branched or cyclic, optionally wherein one or more C of the aliphatic group is replaced by —O—, —NH—, —O—C(O)—, or —C(O)—O—;
wherein the aliphatic group optionally comprises at least one substituent —C(O)OH;
wherein n is an integer from 1 to 3; and
wherein each a and b is independently an integer from 1 to 2.

The aliphatic group can comprise further substituents whose presence does not interfere with the polymerization reaction, or the preparation and use of activated polymers prepared from this polymer as outlined above. For example, one or more C of the aliphatic group can comprise at least one substituent selected from hydroxyl, methyl ester, or methoxy; and/or, one or more C of the aliphatic group can be —C(O)—.

In another embodiment, D′ is an optionally substituted C₃ to C₉ alkylene group that is linear, branched or cyclic. In yet another embodiment, D′ is —(CR₄R₅)—(CR₄R₅)—(CR₄R₅)—, wherein each R₄ and each R₅ is independently selected from —H and —C(O)OH.

In another embodiment, A′ is selected from:

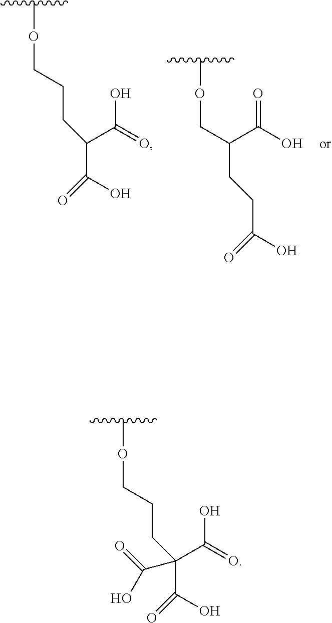 [SMILES: *OCCCC(C(=O)O)C(=O)O ; *OCC(CCC(=O)O)C(=O)O ; *OCCCC(C(=O)O)(C(=O)O)C(=O)O]

In another embodiment, A′ is selected from:

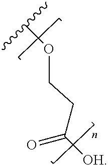

In still yet another embodiment, the polymer comprises a structure of formula (iii):

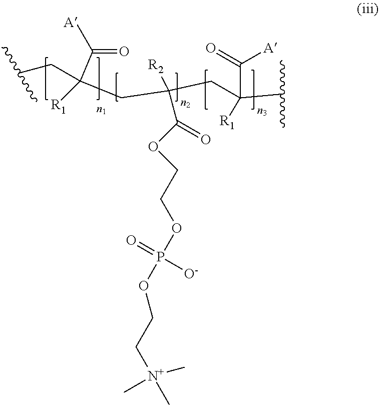

wherein A R₁, and R₂ are as defined above;
wherein each of n₁, n₂, and n₃ is independently selected from an integer from 1 to 2800, preferably wherein each of n₁, n₂, and n₃≥4;
wherein the polymer is prepared by reverse addition-fragmentation chain transfer polymerization (RAFT) controlled radical polymerization.

In another embodiment, there is provided an activated biocompatible polymer for use as a medical adhesive, wherein the activated biocompatible polymer is prepared by a process comprising:
obtaining the polymer or the salt or solvate thereof comprising the structure of formula (iii);
converting the at least one —C(O)OH group to an activated ester group; and
optionally, isolating the activated biocompatible polymer or a physiologically acceptable salt or solvate thereof.

In another embodiment, each of the activated ester groups comprises an N-hydroxysuccinimide ester, such as O—(N-hydroxysucccinimide) ester or O—(N-hydroxysulfosuccinimide) ester; or, a triazine ester, such as an O-(4,6-dimethoxyl-1,3,5-triazin-2-yl) ester. In yet another embodiment, each of the at least one activated ester groups comprises an O-(4,6-dimethoxyl-1,3,5-triazin-2-yl) ester.

In another embodiment, the activated biocompatible polymer has a number average molecular weight (Mₙ) of from about 5.9 kDa to about 4 MDa.

In still another embodiment, the invention provides a medical adhesive composition comprising an activated polymer as defined herein. In embodiments wherein the medical adhesive composition comprises an activated polymer comprising one or more monomer subunits of formula (ia′) having a single activated ester-containing group, the polymer is prepared by RAFT polymerization or other living polymerization.

In another embodiment, the activated biocompatible polymers or compositions containing such activated polymers are applied to biological systems in vivo to facilitate wound healing or anchoring of e.g. collagen-containing medical devices.

The activated biocompatible polymers and precursors thereof described herein are not limited by their method of preparation and can be prepared by any number of conventional synthetic methods, including but not limited to free radical polymerization (FRP), or reverse addition-fragmentation chain transfer polymerization (RAFT) or other types of living polymerization of the respective monomers. The resulting polymers can have a random or organized sequence, and can have any one of a variety of architectures, such as linear, branched architectures, or combinations thereof.

In one embodiment, the activated biocompatible polymers and precursors thereof can be prepared by FRP. A representative FRP synthetic scheme for preparation of exemplary polymer precursors is shown below in Scheme 1. In the following scheme, n is 1.

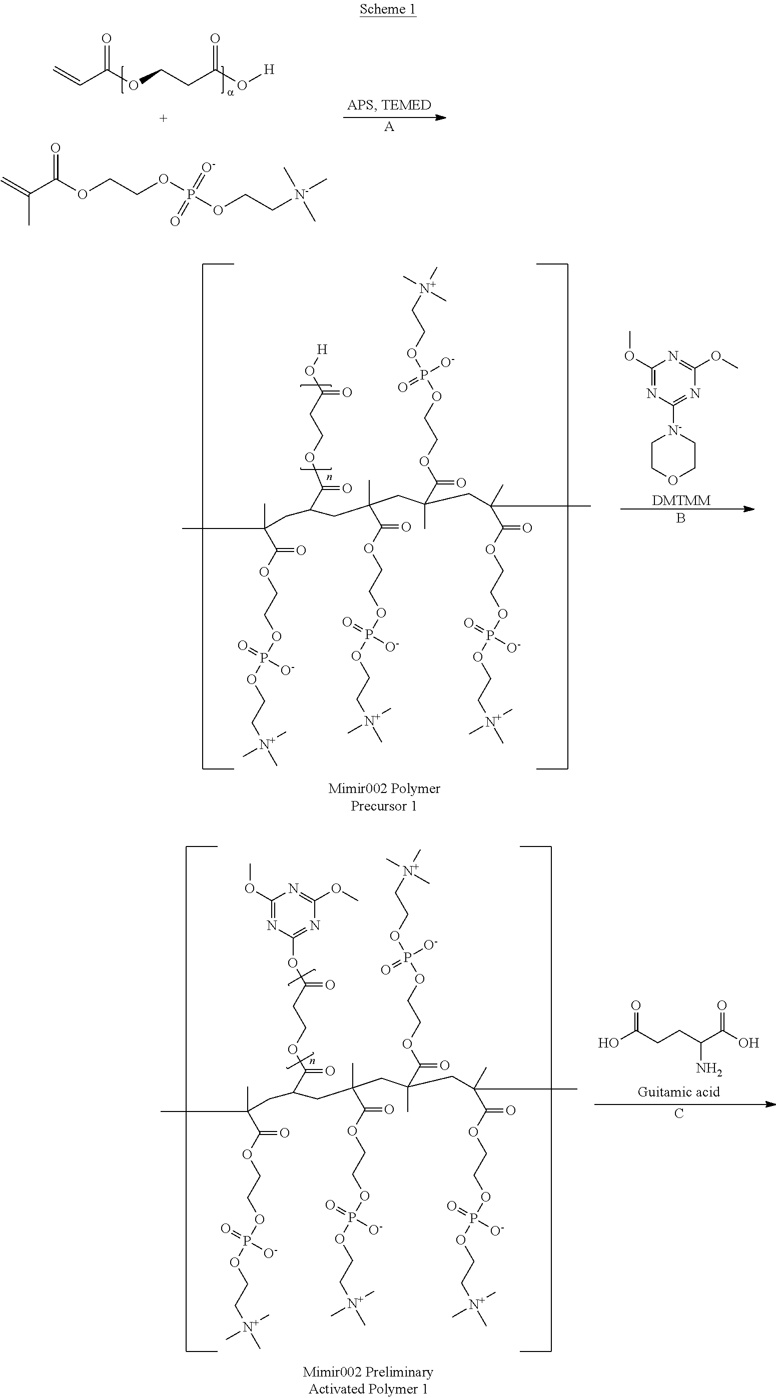

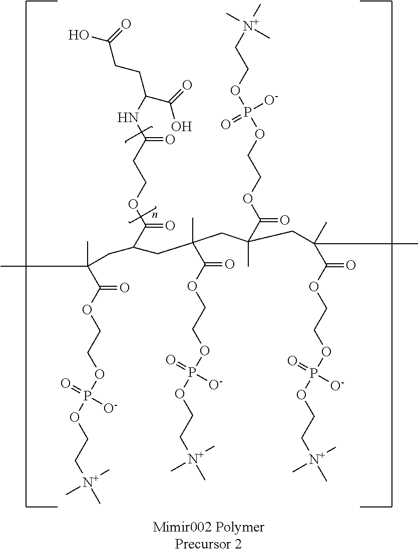

In step A, 2-Carboxyethyl acrylate oligomers (where n is an integer from 1 to 3) and 2-Methacryloyloxyethyl phosphorylcholine are polymerized in the presence of ammonium persulfate (APS) and tetramethylethylenediamine (TEMED) to form Mimir002 Polymer Precursor 1. Those of skill in the art will appreciate that any radical forming entity that is soluble in water and does not immediately degrade further can be used—e.g. 2,2-Azobis[2-(2-imidazolin-2-yl)propane]dihydrochloride (cas: 27776-21-2), 2,2′-Azobis(2-methylpropionitrile), and also photoinitiators are a class of radical forming species that are very often compatible with FRP in water/hydrophilic systems. In one embodiment, the ratio of 2-Carboxyethyl acrylate oligomers to 2-Methacryloyloxyethyl phosphorylcholine can range from 4:1 to 1:15. For the purposes of the present scheme, a ratio of 1:11 is used. The product, Mimir002 Polymer Precursor 1, can be isolated from the starting materials e.g. by dialysis prior to the subsequent reaction.

The carboxylic acid groups of Mimir002 Polymer Precursor 1 are subsequently activated in step B through reaction with 4-(4,6-Dimethoxy-1,3,5-triazin-2-yl)-4-methylmorpholinium chloride (DMTMM) to form Mimir002 Preliminary Activated Polymer 1, which is then coupled to glutamic acid in step C to produce Mimir002 Polymer Precursor 2. The product, Mimir002 Polymer Precursor 2, can be isolated from the starting materials e.g. by dialysis prior to the subsequent reaction.

As will be appreciated by the skilled worker, the structure of Mimir002 Polymer Precursor 2 that is shown is an exemplary structure where a stoichiometric ratio of DMTMM to free carboxylic groups of Mimir002 Polymer Precursor 1 may be present. In other embodiments, including where DMTMM is present in excess relative to the number of free carboxylic groups of Mimir002 Polymer Precursor 1, it is possible for coupling between glutamic acid molecules to occur before or after coupling of an α-amino group of glutamic acid with an activated ester of Mimir002 Polymer Precursor 1. Such coupling between glutamic acid molecules can result in formation of a poly(amino acid) group that is coupled to Mimir002 Polymer Precursor 1, resulting in a more complex, branched polymer architecture. The poly(amino acid) group can have one or more peptide bonds and/or one or more isopeptide bonds. Those of skill in the art will further appreciate that alternative amino acids having carboxylic acids in their side chain, such as aspartic acid, can equivalently be used. Such amino acids can be L-amino acids, D-amino acids, or combinations thereof, such as racemic mixtures.

Mimir002 Polymer Precursor 2 is then reacted with 4-(4,6-Dimethoxy-1,3,5-triazin-2-yl)-4-methylmorpholinium chloride to form the final biocompatible activated polymer Mimir002:

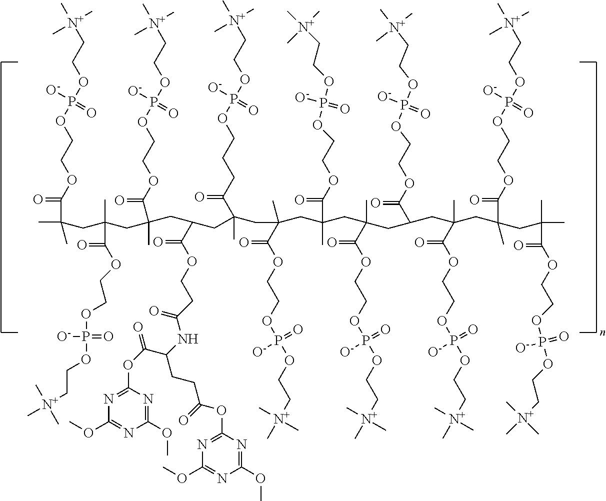

As can be seen, the exemplary biocompatible activated polymer Mimir002 shown above contains a ratio of 2:11 triazine esters to phosphoryicholine residues. The number average molecular weight (Mn) of the biocompatible activated polymer can vary and can range from about 100 kDa to about 4 MDa.

In some embodiments, the biocompatible activated polymer can be purified, such as by size exclusion chromatography, prior to use in therapeutic applications. In other embodiments, the biocompatible activated polymer is used in therapeutic applications without further purification.

Those of skill in the art will appreciate that other agents could be used in place of DMTMM to form activated esters, such as reaction of Mimir002 Polymer Precursor 1 with 1-ethyl-3 (3-dimethylaminopropyl)carbodiimide (EDC), or other suitable carbodiimide crosslinking reagent, and N-hydroxysuccinimide (NHS) or N-hydroxysulfosuccinimide (Sulfo-NHS) to create NHS-activated esters. Triazine esters, such as O-(4,6-dimethoxyl-1,3,5-triazin-2-yl) esters, are particularly preferred as these have been found to be more resistant to hydrolysis than NHS-activated esters, and DMTMM also avoids the use of toxic carbodiimide crosslinking reagents.

A key characteristic of the polymer precursors and biocompatible activated polymer prepared by FRP is the branched structure that is present on the monomer subunits of formula (ia) and formula (iia) as noted above, whereby the polymer precursors have monomer subunits of formula (ia) having at least two —C(O)OH groups and the resulting biocompatible activated polymer has monomer subunits of formula (iia) having at least two activated ester groups. The presence of at least two activated ester groups in the monomer subunit of formula (iia) provides improved functioning of the biocompatible activated polymer as a bonding agent, cross-linking agent, and/or as a glue relative to polymers having only a single activated ester group present in the monomer subunit.

In the above Scheme 1, the 2-Carboxyethyl acrylate oligomers are coupled with an amino acid post-polymerization in steps B and C in order for the polymer precursor (Mimir002 Polymer Precursor 2) to have monomer subunits (i.e. of formula (ia)) having at least two —C(O)OH groups and for the resulting biocompatible activated polymer to have monomer subunits (i.e. of formula (iia)) having at least two activated ester groups. The skilled worker will appreciate that in embodiments where the starting monomers that form the basis of monomer subunits of formula (ia) already have at least two —C(O)OH groups, subsequent coupling to an amino acid having at least two carboxylic acid groups (such as aspartic acid or glutamic acid) will not be required prior to formation of the activated polymer product in order to produce a biocompatible activated polymer having monomer subunits (i.e. of formula (iia)) comprising at least two activated ester groups. Examples of monomers containing more than one carboxylic acid for production of activated polymer product and precursors thereof include:

### 2-[3-(2-Methylprop-2-enoyloxy)propyl]propanedioic acid

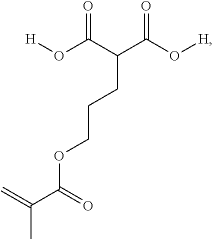

### 2-(2-Methylprop-2-enoyloxymethyl)pentanedioic acid

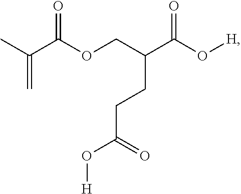

and

### 4-(2-Methyl prop-2-enoyloxy)butane-1,1,1-tricarboxylic acid

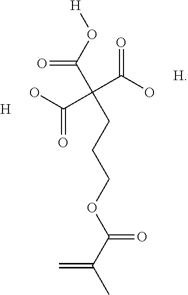

As will also be known to the skilled worker, steps B and C can be included as desired even where the starting monomers that form the basis of monomer subunits of formula (ia) already have at least two —C(O)OH groups, in order to increase the size and to modify the architecture of the final biocompatible activated polymer product.

Further details around the synthesis of the above-noted polymer precursors Mimir002 Polymer Precursor 1 and Mimir002 Polymer Precursor 2, and the final biocompatible activated polymer product Mimir002, are provided in Example 1.

As noted above, the polymers described herein can also be prepared using RAFT polymerization. An exemplary synthetic scheme is shown below in Scheme 2, which uses the reaction materials 2-Carboxyethyl acrylate oligomers (CEAO); 2,2′-Azobis[2-(2-imidazolin-2-yl)propane] Dihydrochloride (AIPD); 4-((((2-Carboxyethyl)thio)carbonothioyl)thio)-4-cyanopentanoic acid (CTCA); and 2-Methacryloyloxyethyl phosphorylcholine (MPC):

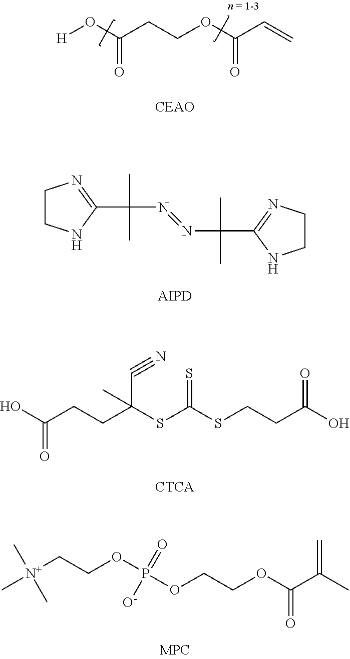

As those of skill in the art will appreciate, alternate radical source/activators such as APS and TEMED can also be used (see Example 6).

Exemplary experimental details are provided below in Example 5. Example 6 also provides an alternate protocol for preparation of activated polymer via RAFT polymerization.

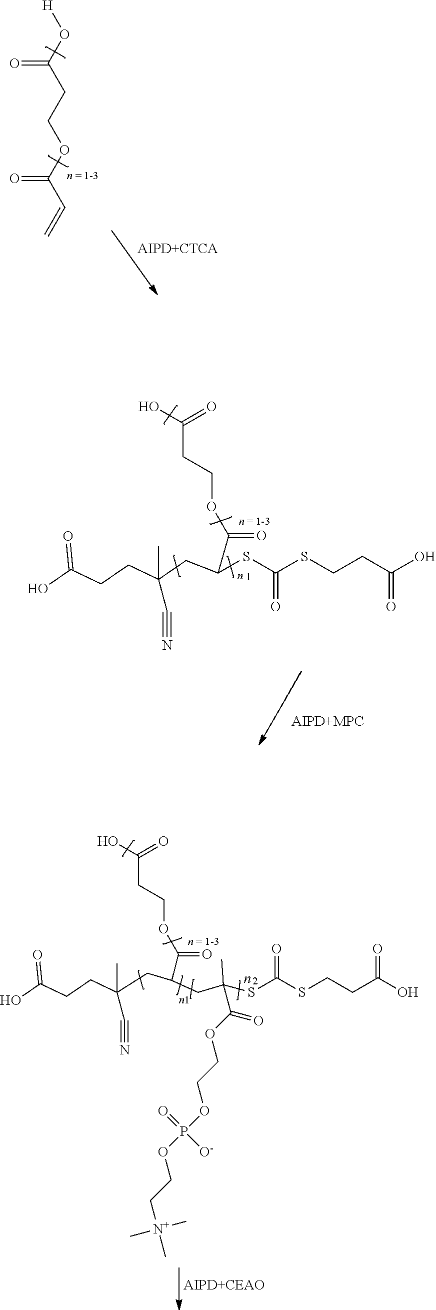

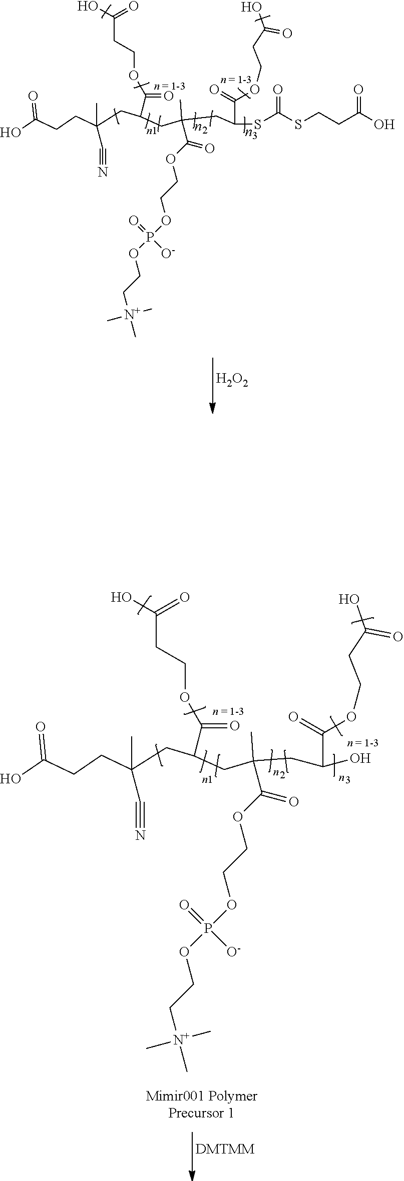

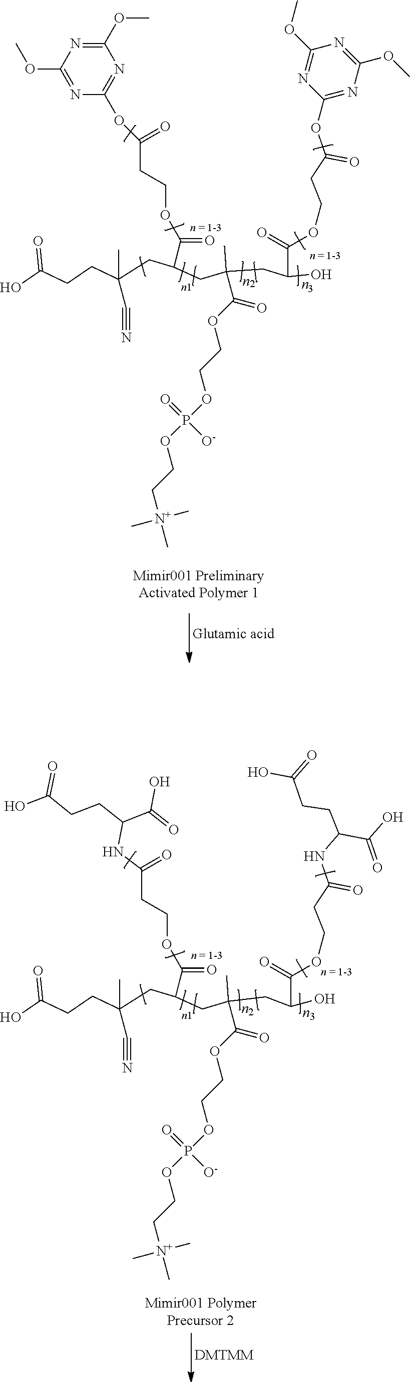

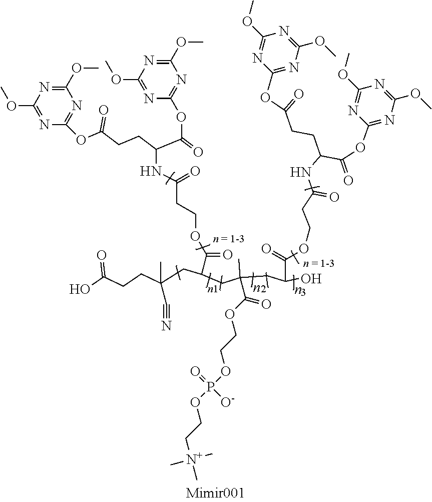

In Scheme 2, n₁, n₂, and n₃ can be such that the number average molecular weight (Mn) of the biocompatible activated polymer is in the same range as noted above—i.e. from about 100 kDa to about 4 MDa. In another embodiment, the number average molecular weight (Mn) of the biocompatible activated polymer can range from about 5.9 kDa to about 4 MDa, or from about 5.9 kDa to about 400 kDa. In one embodiment, each of n₁, n₂, and n₃ is independently selected from an integer from 1 to 2800. In another embodiment, each of n₁, n₂, and n₃≥4.

As noted in respect of the FRP synthesis Scheme 1 noted above, the skilled worker will appreciate that in embodiments where the starting monomers that form the basis of monomer subunits of formula (iia) already have at least two —C(O)OH groups, subsequent coupling to an amino acid having at least two carboxylic acid groups (such as aspartic acid or glutamic acid) will not be required prior to formation of the activated polymer product in order to produce a biocompatible activated polymer having monomer subunits (i.e. of formula (iia)) comprising at least two activated ester groups. In embodiments where the biocompatible activated polymers as defined herein are prepared by FRP and used in medical adhesives, the number of esters on average is ideally at least 2 per 15 phosphorylcholine residues over the full length of the polymer and/or the number of active esters is ideally on average above 0.3 per 1000 Da over the full length of the polymer.

Further, for biocompatible activated polymers prepared by RAFT polymerization, in embodiments where each of n₁, n₂, and n₃ are, for example, ≥4, the preliminary activated polymers having a single activated ester group per monomer subunit (e.g. Mimir001 Preliminary Activated Polymer 1) have utility as a bonding agent, cross-linking agent, and/or as a glue, and the presence of two activated ester groups per monomer subunit and/or coupling step with glutamine is not required. RAFT polymerization methods of producing the polymers disclosed herein are particularly advantageous as they allow for control and predictability over the activated ester group-containing polymer products (and precursors thereof). In embodiments where the biocompatible activated polymers as defined herein are prepared by RAFT (or other living polymerizations) and used in medical adhesives, ideally 1 or more ester is present at each integrated monomer in the regions adjacent to the leading and tailing ends of the polymer and/or the number of active esters are ideally on average above 1 per 600 Da in the regions adjacent to the leading and tailing ends of the polymer (the leading and tailing ends of the polymer being the regions where the backbone is formed from the 4 first and the 4 last integrated monomer moieties),

### Examples

### Materials

The following materials were used in the examples outlined herein: MOPS—4-morpholinepropanesulfonic acid (Sigma-Aldrich, M1254); MES—2-(4-Morpholino)ethane Sulfonic Acid (Fisher Scientific, BP300-100); Tris—Tris(hydroxymethyl)aminomethane (Fisher Scientific, AAJ65594A1); HCl—HCl (Fisher Scientific, 60047420); NaOH—NaOH (Fisher Scientific, AAA1603736); CEAO—2-Carboxyethyl acrylate oligomers (Sigma-Aldrich, 407585); MPC—2-Methacryloyloxyethyl phosphorylcholine (Sigma-Aldrich, 730114); Sodium Nitrate (Fisher Scientific, S342-3); DMTMM—4-(4,6-Dimethoxy-1,3,5-triazin-2-yl)-4-methylmorpholinium chloride (Fisher Scientific, AC348960050); TEMED—N,N,N,N′-Tetramethyl ethylenediamine (Sigma-Aldrich, T22500); APS—Ammonium persulfate (Sigma-Aldrich, 248614); Glutamic acid—L-Glutamic Acid (Fisher Scientific, AAA1250530); AIPD—2,2′-Azobis[2-(2-imidazolin-2-yl)propane] Dihydrochloride (Fisher Scientific); CTCA—4-((((2-Carboxyethyl)thio)carbonothioyl)thio)-4-cyanopentanoic acid (Sigma Aldrich); Acetate buffer (Fisher Scientific); Acetone (Fisher Scientific); ddH₂O—double-distilled water; Gelatin (Sigma-Aldrich, G1890-100G), Low Endotoxin NMP Collagen PS (Nippi Inc., 892 262), Hydrogen Peroxide 30% (Sigma-Aldrich, 216763-100ML). Heat denatured collagen was prepared as described in the protocol from the Low Endotoxin NMP Collagen PS.

### Example 1—Preparation of Activated Polymer Via Free Radical Polymerization

100 mL of Tris buffer (0.5M, pH 7.0) was measured into a 250 mL round bottom flask. The Tris buffer was sonicated and nitrogen flushed for 5 minutes to de-gas. Nitrogen flushing was continued uninterrupted for the duration of the experiment. The reaction vessel was carefully moved from the sonicator bath to a heating sleeve and stirring was started. MPC (2.953 g, 10 mmol) was weighed on weighing paper and added to the reaction vessel. CEAO (110 μL, 750 μmol) was added to the reaction vessel. of APS (85.6 mg, 375 μmol) was weighed on weighing paper and added to the reaction vessel. TEMED (56.2 μL, 377 μmol) was added to the reaction vessel. The reaction was nitrogen flushed continuously under stirring at 20° C. for 4 hours.

The product was dialyzed for 3 days with daily water changes according to:
1) Dialysis tubing was cut to size (11 cm long strip of Spectra/Por 3 Dialysis Membrane, MWCO 3.5 kDa, 54 mm)
2) The dialysis tubing was soaked in 100 mL of ddH2O to remove conservatives.
3) A 2 L cylinder was filled with ddH2O
4) A stir-bar was added to the cylinder and the cylinder was placed on a stir-plate
5) The bottom of the dialysis tube was clamped with a weighted dialysis clamp
6) The polymerization product from the previous step was carefully poured into the tubing
7) The top of the dialysis tube was clamped, and the tube was placed in the ddH2O in the cylinder
8) The dialysis was allowed to progress for 72 hours with water changes every 24 hours.

The dialysed polymerization product (“Mimir002 Polymer Precursor 1”) was collected in a sterile 250 mL beaker.

DMTMM (270 mg, 975.712 μmol) was weighed into a 5 mL microtube. L-Glutamic acid (551 mg, 3.0 mmol) was weighed into a 5 mL microtube. 100 mL of Mimir002 Polymer Precursor 1 was moved to a clean 250 mL round bottom reaction flask. A magnet was added to the round bottom flask and rapid stirring was started. 25 mL of 0.5M MOPS pH 6.8 was added to Mimir002 precursor 1 flask to buffer with 0.1M MOPS. The previously weighed DMTMM was added to the round bottom flask by transferring a small volume of the solution of the flask to the microtube, dissolving by inverting the tube, and pipetting the solution back to the round bottom flask. The solution was allowed to react at 20° C. with stirring for 30 min.

The previously weighed L-Glutamic acid was added to the round bottom flask with the same method that had been used for the DMTMM. The solution was incubated for 30 min. The solution now containing the modified polymer (“Mimir002 Polymer Precursor 2”) was moved to a pre-wet Spectra/Por 3.5 kD dialysis tube, and dialysis was performed in the same manner as previously described.

The Mimir002 Polymer Precursor 2 solution was measured to 137 mL. 34 mL of 0.5 M MOPS was added to make 0.1M MOPS. The resulting solution was mixed through repeated inversion. 1 g (3.61 mmol) of DMTMM was added to form the biocompatible activated polymer (“Mimir002”). The resulting solution containing Mimir002 was placed in 2 clean borosilicate dishes. The solution was placed in a lyophilizer and program (Table 1) was started.

TABLE 1
Lyophilization program

| | Ramp Rate | Shelf Temp | Time | Vacuum | Total Step |
|---|---|---|---|---|---|
| Step | (° C./min) | (° C.) | (hh:mm) | (mBar) | Time |
| 1 | 1 | −40 | 03:00 | | 03:00 |
| 2 | 0.05 | 0 | 06:00 | 0.05 | 19:20 |
| 3 | 0.05 | 30 | 06:00 | 0.05 | 12:00 |
| 4 | 1 | 24 | ∞ | | |

Approximately 7.5 g of lyophilized materials was obtained, of which about 40% was determined to be polymerization product, taking into account the amounts of MOPS and DMTMM present, assuming near 100% reaction efficiency of previous steps (which has consistently been obtained in other experiments). Further purification of the polymer is possible using size exclusion chromatography, but not necessary in this case as both MOPS and DMTMM can be used in downstream applications. In particular, the presence of MOPS and DMTMM aids and creates a higher degree of crosslinking and helps control the pH in those applications. As larger-scale purification via size exclusion chromatography is expensive, it is particularly advantageous that the polymer can be used for downstream applications without further purification.

### Example 2—Characterization of Activated Polymer via Size Exclusion Chromatography (SEC)/Gel Permeation Chromatography (GPC)

Size Exclusion Chromatography (SEC)/Gel Permeation Chromatography (GPC) was carried out to determine the size of the activated polymer, Mimir002. The Size Exclusion Chromatography (SEC)/Gel Permeation Chromatography (GPC) system was equipped with an oven that houses three detectors: Refractive Index (RI), Right Angle and Low Angle Light Scattering (RALS/LALS), and Four-Capillary Differential Viscometer.

In Triple Detection SEC/GPC, refractive index (RI) detector is employed to calculate concentration, refractive index increment (dn/dc), and injection recovery of polymer solutions. A UV detector is employed to calculate the concentration of UV absorbing material and the UV extinction coefficient (dA/dc). Light scattering provides absolute molecular weight and radius of gyration while the viscometer delivers intrinsic viscosity, hydrodynamic radius, and chain conformational and structural parameters (i.e. branching).

The SEC/GPO method was carried out as follows:
the lyophilized sample containing Mimir002 was dissolved in 0.3M Sodium Nitrate at a concentration of approximately 1 mg/mL
the sample was left overnight on a rocker to dissolve;
the sample was filtered through a 0.22 urn nylon syringe filter before injection; and
the details of the run settings and column details are noted in Table 2.

TABLE 2
GPC method setup

| | |
|---|---|
| Instrument | Viscotek TDA305 and GPCmax |
| Detectors | Refractive Index, Light Scattering, and Viscometer |
| Mobile Phase | 0.3M Sodium Nitrate mixture solution in HPLC |
| | grade water |
| SEC/GPC | GMPWXL × 2 |
| Columns | |
| Flow Rate | 0.7 mL/min |
| Analysis | 30° C. |
| Temperature | |
| Analysis Software | OmniSEC 4.6.2 |
| Calibration Type | Triple Detection |
| Calibration | PATD-PEG20K (PEG20K refers to polyethylene |
| Standard | glycol (PEG) with MW = 20K) |
| Verification | PATD-DXT72K (DXT72K refers to dextran |
| Standard | with MW = 72K) |
| Concentration of | 3 mg/mL |
| the Standards | |
| dn/dc of the | 0.132 mL/g for PEG 20K, and 0.147 mL/g for |
| Standards | DXT72K |
| Refractive Index | 1.333 |
| of Solvent | |
| Injections per | 3/sample |
| Sample | |

### Results

The molecular weight (Mw) was determined to be 2.4 MDa and the number average molecular weight (Mn) was found to be 1.5 MDa (Table 3), The polydispersity index (PDI) was calculated to be 1.6 and the hydrodynamic radius was measured at 33.8 nm (Rh (nm)).

TABLE 3
Characterization using SEC/GPC

| | | | | | | % |
|---|---|---|---|---|---|---|
| Injection | 1 | 2 | 3 | Average | σ (±) | RSD |
| dn/dc (mL/g) | 0.107 | | | | | |
| Vp (mL) | 13.68 | 13.69 | 13.69 | 13.69 | 0 | 0.0003 |
| Mn (Da) | 1589000 | 1445000 | 1530000 | 1521333 | 59106 | 0.0389 |
| Mw (Da) | 2446000 | 2367000 | 2428000 | 2413667 | 33807 | 0.014 |
| Mz (Da) | 3610000 | 3431000 | 3728000 | 3589667 | 122099 | 0.034 |
| Mp (Da) | 2340000 | 2303000 | 2371000 | 2338000 | 27797 | 0.0119 |
| Mw/Mn (PDI) | 1.539 | 1.638 | 1.587 | 1.588 | 0.04 | 0.0255 |
| [η] | 1.1575 | 1.1102 | 1.145 | 1.1376 | 0.02 | 0.0176 |
| (dl/g) | | | | | | |
| Rh (nm) | 34.26 | 33.34 | 33.97 | 33.85 | 0.38 | 0.0113 |
| M − H | −0.665 | −0.707 | −0.734 | −0.702 | 0.028 | −0.0404 |
| α | | | | | | |
| M − H | 4.326 | 4.574 | 4.749 | 4.55 | 0.174 | 0.0381 |
| Log K | | | | | | |

Mz: Z-average molar mass / third moment or third power average molar mass
Vp: Pore volume / working resolving range
η: Intrinsic viscocity (measured in dl/g or deciliters per gram)
Mp: peak molecular weight
M − H: Mark-Houwink a and k. Values related to the solvent system, used in calculating the molecular weight based on intrinsic viscosity

### Example 3—Functional Crosslinking Study

A functional study of the activated polymer Mimir002 from Example 1 as a crosslinker in hydrogel was performed and gelation speed was measured. 200 μL of 20% Mimir002 mixture (40% of dry weight is polymer, 8% polymer in this solution) was prepared in a 1 mL syringe from the lyophilized powder from the synthesis protocol above. 200 μL of 20% w/w heat denatured collagen (i.e. gelatin) was prepared in a 1 mL syringe. 200 μL of 6% w/w collagen was prepared in a 1 mL syringe. All solutions mentioned were in 100 mM MOPS buffer in ddH₂O pH adjusted using 6N HCl.

The prepared collagen and gelatin were mixed through a female to female luer connector to form a solution.

The collagen and denatured collagen (gelatin) solution was mixed with the Mimir002 solution by shuttling the mixture between the two syringes through a luer connector 80 times and subsequently ejecting the combined mixture to a clean glass surface.

The material was manually manipulated briefly every 60 sec until full crosslinking of the material was observed.

### Results

The material hardened over 4 min. The material lost tackiness/stickiness over 10 minutes indicating progression of crosslinking. The material was not fully crosslinked at 20 min as it had not reached its final consistency. At 60 minutes the material was a densely crosslinked hydrogel, and no further changes were observed over the following 24 hours.

### Example 4—Animal Study of Adhesion and Healing

Materials prepared using Mimir002 activated polymer prepared in Example 1 were compared to a control material in which DMTMM (1% w/v) was used instead of Mimir002.

TransBIOTech animal care facility is accredited by the Canadian Council on Animal Care (CCAC). This study was approved by the Cégep de Lévis Animal Care Committee and complied with CACC standards and regulations governing the use of animals for research.

Male Sprague Dawley rats (Charles River, Rayleigh, USA), weighing 225-250 g at delivery were used for this study. Following arrival in the animal facility, all animals were subjected to a general health evaluation. An acclimation period of 5-7 days was allowed before the beginning of the study.

The animals were housed under standardized environmental conditions. The rats were housed in auto-ventilated cages, 1 per cage to prevent grooming between rats. Each cage was equipped with a manual water distribution system. A standard certified commercial rodent diet was provided ad libitum for the acclimation phase. Water was provided ad libitum at all times. It is considered that there are no known contaminants in the diet and water that would interfere with the objectives of the study. Each cage was identified for the corresponding group, indicating the treatment and the identity of the animals housed in the cage.

The animal room was maintained at a controlled temperature of 21.0±1° C. and a relative humidity of 40±10%. A controlled lighting system assured 12 hours light; 12 hours dark per day to the animals. Adequate ventilation of 18-20 air changes per hour was maintained.

24 hours before the formation of the wounds, the rats were anesthetised using isoflurane and the backs of the rats were shaved with a razor and depilated with a depilatory cream. Prior to wound formation, 5 ml/kg Ringer's lactate was administered subcutaneously to prevent dehydration. Rats were given a dose of the analgesic buprenorphine SR (1 mg/kg) to prevent procedurally induced pain. The rats were deeply anesthetized by isoflurane inhalation and placed on a heat mat. The skin surface was disinfected with chlorhexidine and using a 12 mm diameter biopsy punch, one wound was made in the upper back of the rat, fully penetrating the skin, but without entering into the underlying muscle.

Either the prototype hydrogels (200 uL of 8% mimir002 mixture containing <1% DMTMM, 200 μL of 20% w/w heat denatured collagen (gelatin), 200 μL of 6% w/w collagen) were applied into the wounds or the control solution (DMTMM (1% w/v), 200 μL of 20% w/w heat denatured collagen (gelatin), 200 μL of 6% w/w collagen) was applied in sufficient quantity to cover the wound, the animals were subsequently removed from anesthesia. All these solutions were adjusted to 100 mM MOPS, and pH 6.5. Then, a non-porous silicon sheet was applied after leaving 15 minutes to dry after polymerization of Mimir002. Wounds were covered with a self-adhesive bandage (VetWrap) to hold the dressings in place.

Thrice (3×) per week, on Mondays, Wednesdays and Fridays, rats were weighed and the self-adhesive bandage was removed to expose the wound and allow pictures to be taken with length scales. This was performed for a period of 14 days.

The animals were sacrificed at 14 days post surgery. Tissues were be collected and split into two different samples, one immediately fixed in formalin and further paraffin-embedded for microtome sectioning and hematoxylin and eosin (H&E) staining. The second sample was placed in 4% paraformaldehyde in phosphate buffer at 4° C. overnight and then incubated in PBS 30% sucrose for 24-72 h before being embedded in OCT-compound:sucrose and cryopreserved by freezing in isopentane. OCT embedded tissues were stored at −80° C.

### Results

Adhesion of the material to the wound bed was very strong and the materials were retained without a covering/bandaged after 24 hours, the vet-wrap was however left in place when the animals weren't actively monitored to decrease the risk of them scratching the wound site.

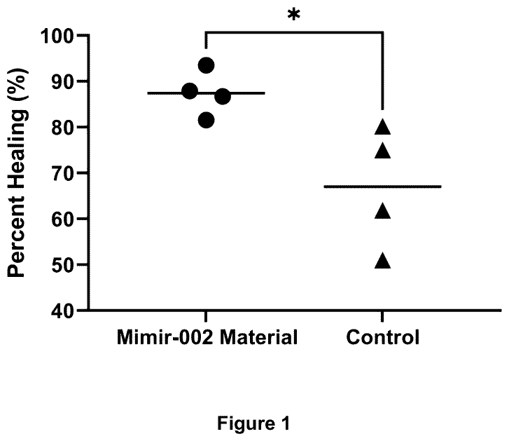

The healing was significantly quicker in the group that was treated with the material containing Mimir002 compared to the controls (20% smaller wound at day 13 post surgery) (FIG. 1).

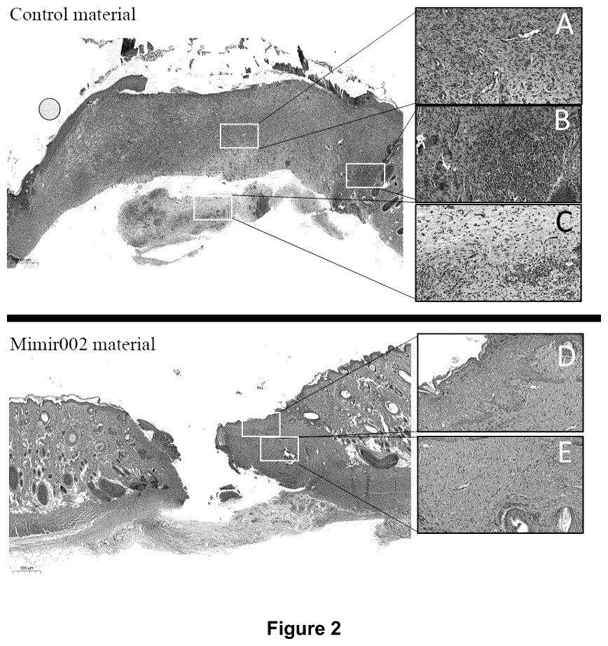

Hematoxylin and eosin staining showed markedly lower lymphocyte infiltration in the tissue regenerating in the Mimir002 material as well as more complete re-epithelialization, and a smaller amount of granulation tissue (FIG. 2.). A higher number of fibroblasts were seen in the control materials compared to the Mimir002 materials, indicative of tissue repair still being high at the day of euthanasia (day 14 post surgery).

### Example 5—Protocol for Preparation of Activated Polymer Via Reverse Addition-Fragmentation Chain Transfer Polymerization (RAFT)

RAFT polymerization of CEAO is performed in aqueous media according to:

CEAO (8.5 g, 50.0 mmol), CTCA (230 mg, 0.75 mmol), AIPD (1.21 mg, 0.38 mmol), and 100 mM acetate buffer (pH=5.0, 75 ml) are placed in a clean round-bottom tube. A large magnetic stirring bar is added. The solution is degassed by sonication and nitrogen gas, nitrogen gas is allowed to bubble through the solution for the full duration of the reaction to ensure 02 levels are kept at a minimum. The reaction is allowed to progress at constant stirring and 45° C. for 20 h.

The reaction mixture is precipitated into an excess of acetone and isolated by filtration to produce dithiocarbamate terminated poly-CEAO as a powder.

Dithiocarbamate terminated poly-CEAO (3.0 g), MPC (22 g, 75.0 mmol), AIPD (1.21 mg, 0.38 mmol), and 100 mM acetate buffer (pH=5.0, 75 ml) are placed in a clean round-bottom tube. A large magnetic stirring bar is added. The solution is degassed by sonication and nitrogen gas, nitrogen gas is allowed to bubble through the solution for the full duration of the reaction to ensure 02 levels are kept at a minimum. The reaction is allowed to progress at constant stirring and 45° C. for 20 h.

The reaction mixture is precipitated into an excess of acetone and isolated by filtration to produce dithiocarbamate terminated poly-CEAO-poly-MPC as a powder.

Dithiocarbamate terminated poly-CEAO-poly-MPC (15 g), CEAO (4 g, 23.5 mmol), AIPD (1.21 mg, 0.38 mmol), and 100 mM acetate buffer (pH=5.0, 75 ml) are placed in a clean round-bottom tube. A large magnetic stirring bar is added. The solution is degassed by sonication and nitrogen gas, nitrogen gas is allowed to bubble through the solution for the full duration of the reaction to ensure 02 levels are kept at a minimum. The reaction is allowed to progress at constant stirring and 45° C. for 20 h.

The terminal dithiocarbamate is removed by addition of H₂O₂.

The reaction mixture is precipitated into an excess of acetone and isolated by filtration to produce poly-CEAO-poly-MPC-poly-CEAO as a powder.

The carboxylic acids are activated in a manner identical to that of Example 1.

### Example 6—Preparation of Activated Polymer via Reverse Addition-Fragmentation Chain Transfer Polymerization (RAFT)

The reaction was performed at 45° C.

200 mL of acetate buffer (0.15M, pH 5.0) was measured into a 250 mL round bottom flask.

Buffer was sonicated and nitrogen flushed for 5 minutes to de-gas.

Sonication was continued for 10 min after all reagents had been added.

Nitrogen flushing was continued uninterrupted for the duration of the experiment.

˜2.931 mL (20 mmol) of CEAC was added to the reaction vessel.

440 mg (1.43 mmol) of CTCA was weighed and added to the reaction vessel.

21.4 μL (143 μmol) of TEMED was added to the reaction vessel.

32.6 mg (143 μmol) of APS was weighed on weighing paper and added to the reaction vessel.

The reaction was nitrogen flushed continuously at 45° C. for >4 hours.

5.905 g (20 mmol) of MPC was added.

21.4 μL of TEMED was added to the reaction vessel.

32.6 mg of APS was weighed on weighing paper and added to the reaction vessel.

The reaction was nitrogen flushed continuously at 45° C. for >4 hours (24).

˜2.931 mL (20 mmol) of CEAO was added to the reaction vessel.

21.4 μL (143 μmol) of TEMED was added to the reaction vessel.

32.6 mg (143 μmol) of APS was weighed on weighing paper and added to the reaction vessel.

The reaction was nitrogen flushed continuously at 45° C. for >4 hours (24).

The reaction was quenched by exposing it to air.

ddH2O was added to the reaction vessel to make the volume up to approximately 200 mL.

10 mL sample was pulled for later nuclear magnetic resonance (NMR) and GPC-SEC analysis.

The solution was heated to 70° C. in air.

0.071 moles of hydrogen peroxide (7.25 mL of 30% sol) was added to the solution at 50 times the molar amount of CTCA.

Cleavage of the terminal dithiocarbamate from the polymers was performed. Heating was maintained at 70° C. The solution was exposed to air but capped to decrease evaporation and risk of contamination.

Heating was resumed for 7 h.

After cleavage of the terminal dithiocarbamate, the product was dialyzed against ddH2O for 72 h on a 3.5 kD membrane with water changes every 24 h.

At the end of dialysis the volume was ˜300 mL.

A 10 mL sample was pulled for later nuclear magnetic resonance (NMR) and GPC-SEC analysis—corresponding to the Mimir001 Polymer Precursor 1 of Scheme 2.

pH was adjusted by addition of 100 mL of 400 mM MES pH 6.3 which brought the pH to 6.2-6.3 range.

DMTMM was added (⅓ of the expected total yield of polymer, 3.650 g) to produce the product—corresponding to Mimir001 Preliminary Activated Polymer 1 of Scheme 2.

The product as well as samples pulled were lyophilized.

The resulting lyo-cake was analyzed using GPC-SEC and nuclear magnetic resonance (NMR).

GPC-SEC was performed as outlined in Example 2. The experimentally determined Mₙ of 10.034 kDa of the Mimir001 Polymer Precursor 1 was within tolerance levels (about 20%) of the expected Mₙ of 8.214 kDa. The higher experimentally determined Mₙ is believed to be due to use of linear polyethylene glycol (PEG) as a calibration standard, which is less bulky compared to the produced polymers. Likewise, the Mimir001 Preliminary Activated Polymer 1 was expected to have an Mₙ ranging between 12.025 kDa and 14.690 kDa, and the experimentally determined Mₙ was higher as compared to these theoretical targets (19.553 kDa). It is expected that use of a more highly branched calibration standard will improve the accuracy of the experimentally determined Mₙ values. The polydispersity was 1.2 which indicates small variance in polymer size.

NMR analysis revealed a loss of peaks between 5.6 and 6.4 PPM relative to the monomer NMR spectra, where these peaks represented the hydrogens close to the terminal alkenes of the acrylic and methacrylic groups of the monomers, evidencing complete reaction and removal of the monomers in the polymer synthesis. In addition, the addition of triazine esters at the carboxylic residues of the polymer was expected to cause a marked decrease of signal at 1 PPM and an increase of a peak right below 3 PPM. This shift in peaks was seen in the experimental data, evidencing the formation of the triazine activated esters in the Mimir001 Preliminary Activated Polymer 1.

Both NMR and GPC-SEC results indicate that the reactions proceeded as expected. The molecular weight of the resultant polymers might be slightly above the ideal reaction assumption, which is always expected as this will be the result of any early loss of chain transfer agent (CTA); any functional reduction in CTA agent shifts the monomer/CTA ratio and will result in higher degree of polymerization. The experimentally obtained Mn values are however most likely slightly higher than real values due to polymer bulkiness. NMR verifies the progression and high efficacy of each protocol step, and is a close fit to predicted spectra.

### Example 7—Crosslinking Study with Activated Polymer of Example 6

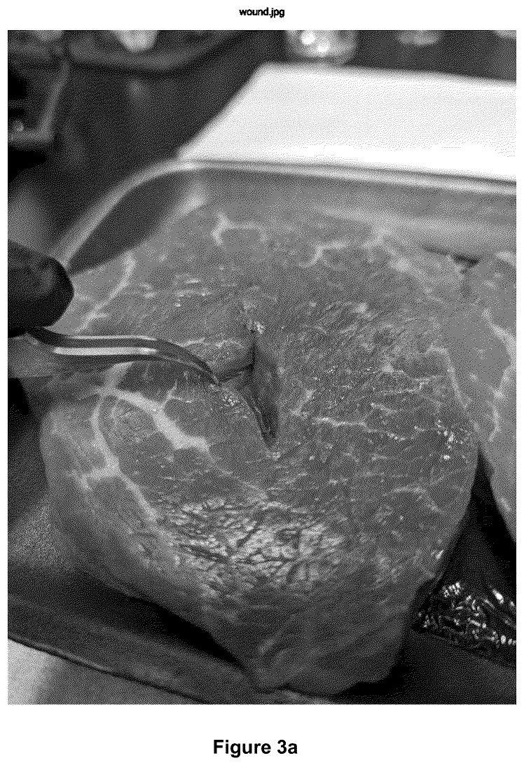

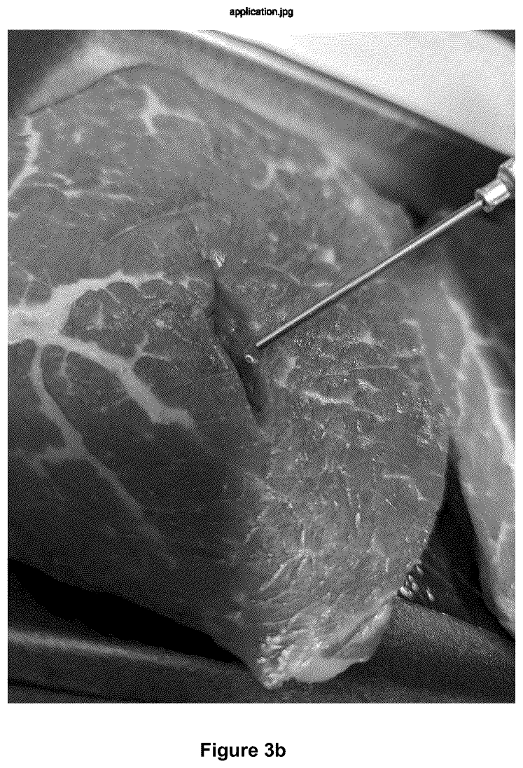

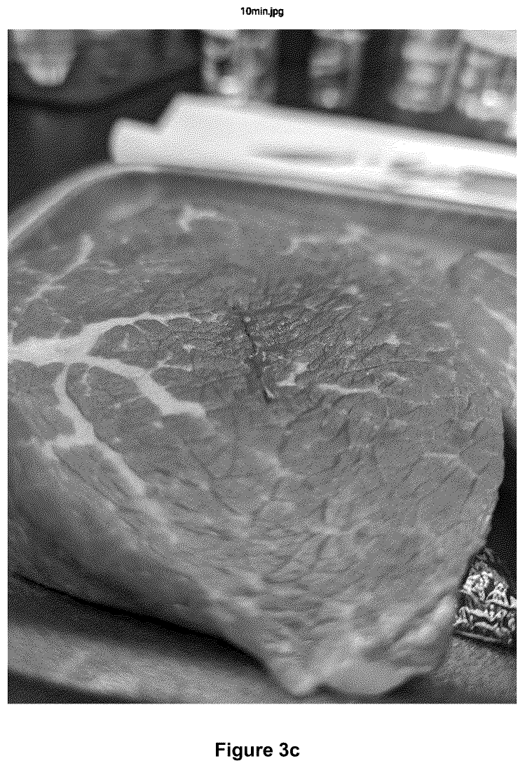

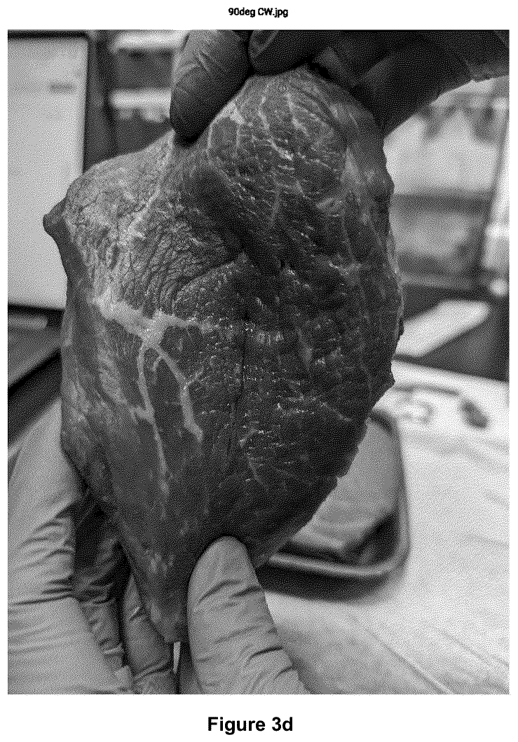

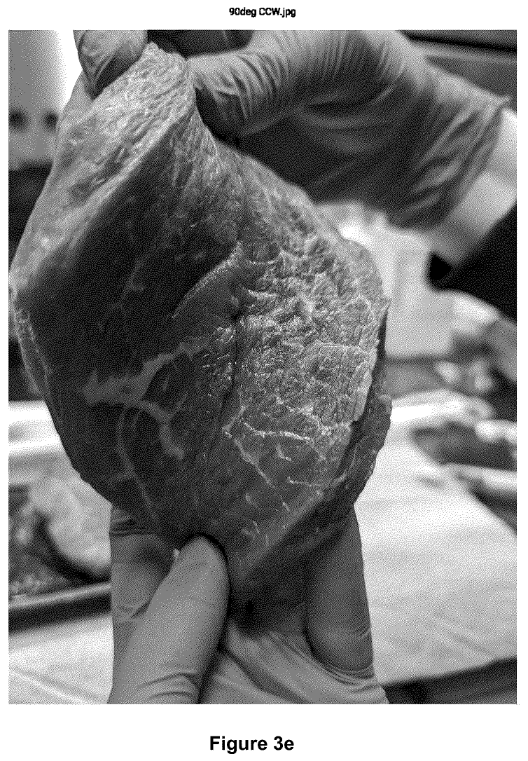

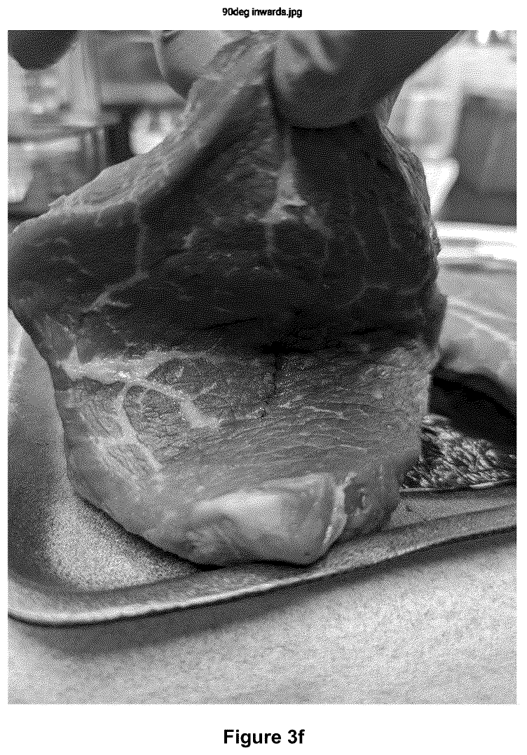

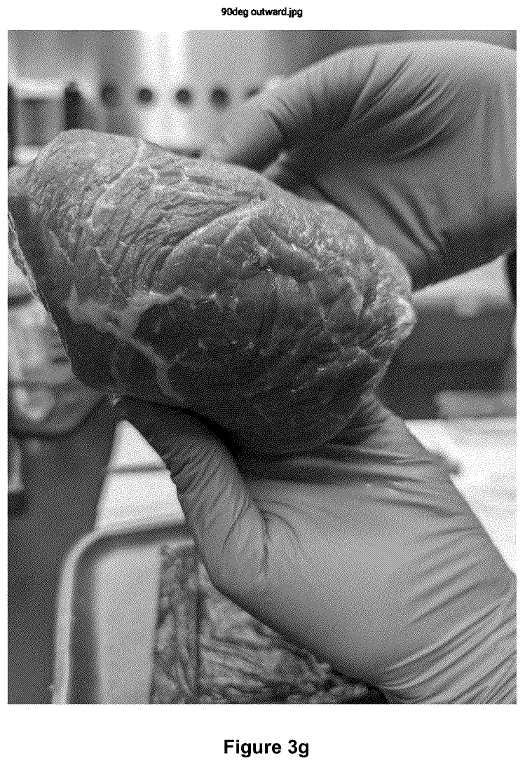

A deep (˜1 cm) cut was prepared in muscle tissue (bovine tissue) using a #10 scalpel (FIG. 3a).

2.152 g of 300 g bloom porcine gelatin was weighed in a 50 mL falcon tube.

Gelatin was dissolved to 20% w/w in MES pH6.3.

MES pH 6.3 was prepared.

Gelatin solution was heated at 80° C. for 30 min.

The pH was measured and found to be 5.6.

The solution was split in 2 into 10 mL henke-ject syringes by aspirating. These aliquots were called aliquot 2 and aliquot 3.

MES pH 6.3 was added in a 1:1 ratio to aliquot 2.

MES pH >6.7 was added in a 1:1 ratio to aliquot 3.

The pH was measured in aliquot 2 and found to be 5.9.

The pH was measured in aliquot 3 and found to be 6.3.

Aliquot 3 was gradually mixed into aliquot 2 until pH 6.0 was reached. (Final ratio aliquot 2/aliquot 3 was 4:1)

Mimir001 Preliminary Activated Polymer 1 was dissolved to 20% w/w in ddH2O.

pH 6.0 gelatin solution was mixed with Mimir001 Preliminary Activated Polymer 1 solution in a 2:1 ratio. Final volume 3 mL.

The cut in the muscle tissue was repaired by ejecting <0.5 mL of the mixture into the wound (FIG. 3b) and pressing the sides of the wound together using surgical forceps. FIG. 3c shows the wound at 10 min after application of the mixture.

A stress test was performed at 10 min:
1. 90° torsion clockwise (FIG. 3d)
2. 90° torsion counter-clockwise (FIG. 3e)
3. 90° fold inwards (FIG. 3f)
4. 90° fold outwards (FIG. 3g)

### Results

Gelation of the material was noticeable at 3 min.

Material was a solid hydrogel at 9 min.

Stress test performed at 10 min as described in methods. No separation from wound edges occurred during the stress test performed at 10 min following application of the adhesive composition.

The material did not shrink or harden further after 20 minutes indicating that full crosslinking had transpired.

All publications, patents and patent applications mentioned in this Specification are indicative of the level of skill of those skilled in the art to which this invention pertains and are herein incorporated by reference to the same extent as if each individual publication, patent, or patent application was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims. The scope of the claims should not be limited to the preferred embodiments set for the description, but should be given the broadest interpretation consistent with the description as a whole.

1-55. (canceled)

56. An activated polymer comprising:
one or more monomer subunits of formula (iia): 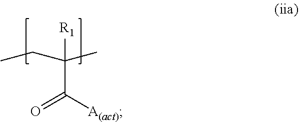
and
one or more monomer subunits of formula (ib): 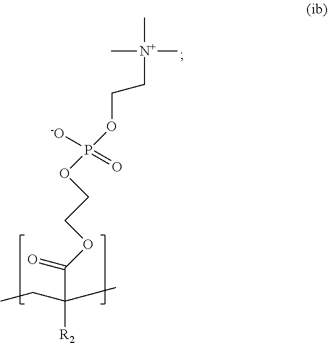
wherein
R₁ and R₂ are independently selected from H or methyl; and
A_{(act)} is an activated ester-containing group comprising at least two activated ester groups;
or a salt or solvate thereof.

57. The activated polymer of claim 56, wherein R₂ is methyl.

58. The activated polymer of claim 56, wherein R₁ is H.

59. The activated polymer of claim 56, wherein the monomer subunits of formula (iia) and the monomer subunits of formula (ib) are present in a ratio of from about 4:1 to about 1:15.

60. The activated polymer of claim 56, wherein A_{(act)} is selected from formula (A_{(act)}−1) or formula (A_{(act)}−2): 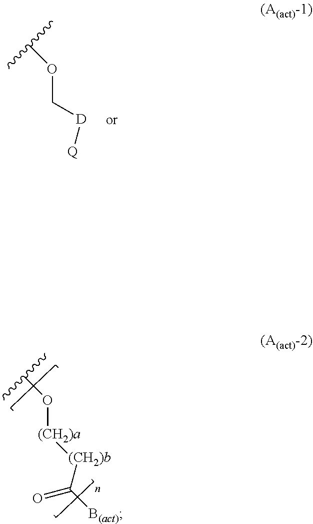
wherein D is a substituted C₃ to C₉ aliphatic group that is linear, branched or cyclic, optionally wherein one or more C of the aliphatic group is replaced by —O—, —NH—, —O—C(O)—, or —C(O)—O—;
wherein the aliphatic group comprises at least one substituent Q;
wherein each B_{(act)} is independently selected from: 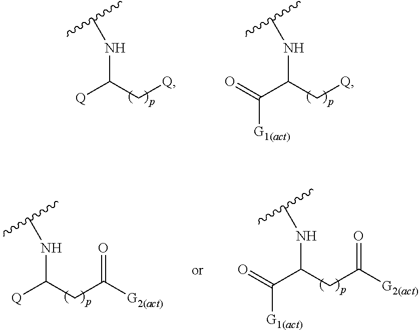
wherein each Q is an activated ester group;
wherein each of G_{1(act)} and G_{2(act)} is independently selected from:
(a) an amino acid residue of the formula G_{3(act)} comprising two activated ester groups Q: 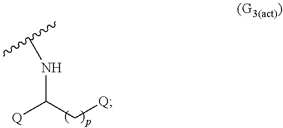
or
(b) a poly(amino acid) group formed from a condensation reaction of an amino acid residue of the formula G₃: 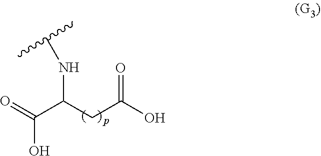
and one or more amino acids of the formula: 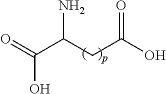
wherein the poly(amino acid) group comprises one or more peptide bonds and/or one or more isopeptide bonds, and wherein the poly(amino acid) group comprises at least two activated ester groups Q;
wherein each activated ester group Q corresponds to one of the at least two activated ester groups of group A_{(act)};
wherein n is an integer from 1 to 3;
wherein each a and b is independently an integer from 1 to 2; and
wherein each p is independently an integer from 1 to 2.

61. The activated polymer of claim 60, wherein D is a substituted C₃ to C₉ alkylene group that is linear, branched or cyclic.

62. A polymer comprising:
one or more monomer subunits of formula (ia): 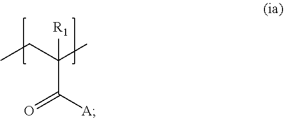
and
one or more monomer subunits of formula (ib): 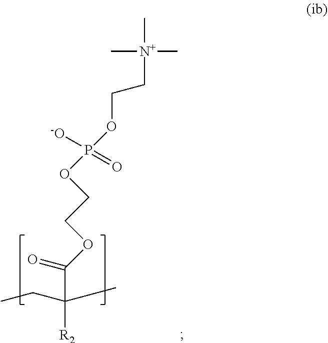
wherein
R₁ and R₂ are independently selected from H or methyl; and
A is a carboxylic acid-containing group comprising at least two —C(O)OH groups;
or a salt or solvate thereof.

63. The polymer of claim 62, wherein the monomer subunits of formula (ia) and the monomer subunits of formula (ib) are present in a ratio of from about 4:1 to about 1:15.

64. The polymer of 62, wherein A is selected from formula (A-1) or formula (A-2): 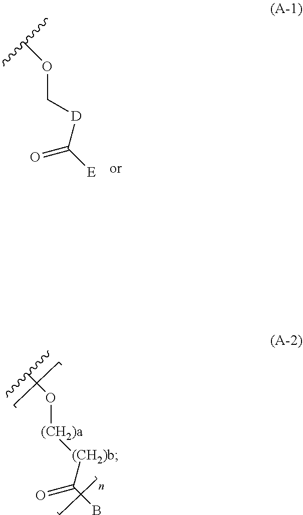
wherein D is a substituted C₃ to C₉ aliphatic group that is linear, branched or cyclic, optionally wherein one or more C of the aliphatic group is replaced by —O—, —NH—, —O—C(O)—, or —C(O)—O—;
wherein the aliphatic group comprises at least one substituent —C(O)E;
wherein each E is independently selected from —OH or B;
wherein each B is independently selected from: 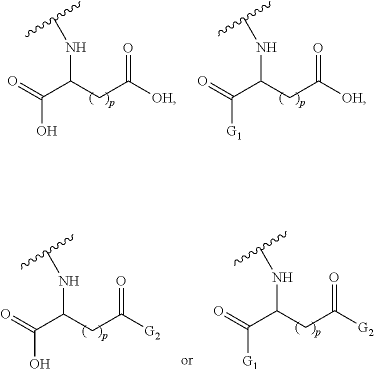
wherein each of G₁ and G₂ is independently selected from:
(a) an amino acid residue of the formula G₃: 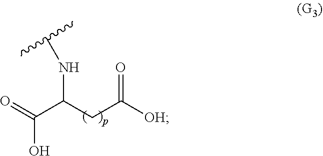
or
(b) a poly(amino acid) group formed from a condensation reaction of G₃ and one or more amino acids of the formula: 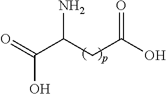
wherein the poly(amino acid) group comprises one or more peptide bonds and/or one or more isopeptide bonds;
wherein n is an integer from 1 to 3;
wherein each a and b is independently an integer from 1 to 2; and
wherein each p is independently an integer from 1 to 2.

65. An activated biocompatible polymer for use as a medical adhesive, wherein the activated biocompatible polymer is prepared by a process comprising:
obtaining the polymer or the salt or solvate thereof of claim 62;
converting the at least two —C(O)OH groups to activated ester groups; and
optionally, isolating the activated biocompatible polymer or a physiologically acceptable salt or solvate thereof.

66. An activated polymer comprising:
one or more monomer subunits of formula (iia′): 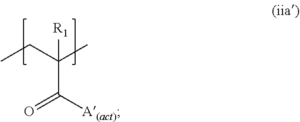
and
one or more monomer subunits of formula (ib): 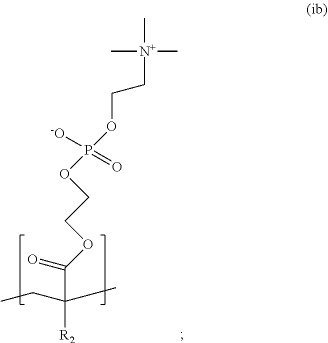
wherein
R₁ and R₂ are independently selected from H or methyl; and
A′_{(act)} is an activated ester-containing group comprising at least one activated ester group;
or a salt or solvate thereof.

67. The activated polymer of claim 66, wherein R₂ is methyl.

68. The activated polymer of claim 66, wherein R₁ is H.

69. The activated polymer of claim 66, wherein the monomer subunits of formula (iia′) and the monomer subunits of formula (ib) are present in a ratio of from about 4:1 to about 1:15.

70. The activated polymer of claim 66, wherein A′_{(act)} is selected from formula (A′_{(act)}−1) or formula (A′_{(act)}−2): 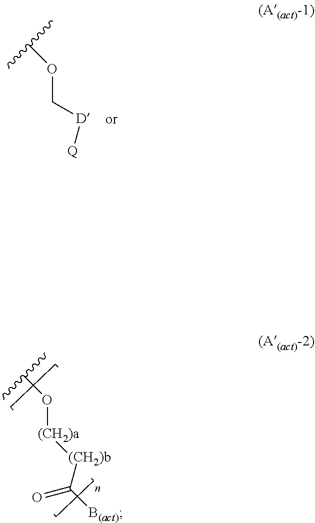
wherein D′ is an optionally substituted C₃ to C₉ aliphatic group that is linear, branched or cyclic, optionally wherein one or more C of the aliphatic group is replaced by —O—, —NH—, —O—C(O)—, or —C(O)—O—;
wherein the aliphatic group optionally comprises at least one substituent Q;
wherein each Q is an activated ester group;
wherein each activated ester group Q corresponds to one of the at least one activated ester groups of group A′_{(act)};
wherein n is an integer from 1 to 3; and
wherein each a and b is independently an integer from 1 to 2.

71. The activated polymer of claim 70, wherein D′ is an optionally substituted C₃ to C₉ alkylene group that is linear, branched or cyclic.

72. A polymer comprising:
one or more monomer subunits of formula (ia′): 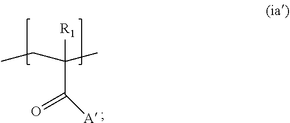
and
one or more monomer subunits of formula (ib): 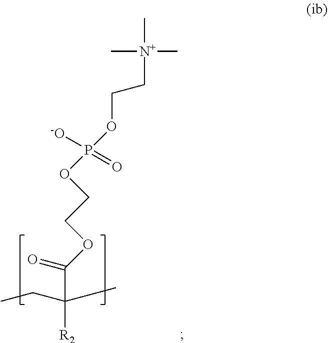
wherein
R₁ and R₂ are independently selected from H or methyl; and
A′ is a carboxylic acid-containing group comprising at least one —C(O)OH group;
or a salt or solvate thereof.

73. The polymer of claim 72, wherein the monomer subunits of formula (ia′) and the monomer subunits of formula (ib) are present in a ratio of from about 4:1 to about 1:15.

74. The polymer of claim 72, wherein A′ is selected from formula (A′-1) or formula (A′-2): 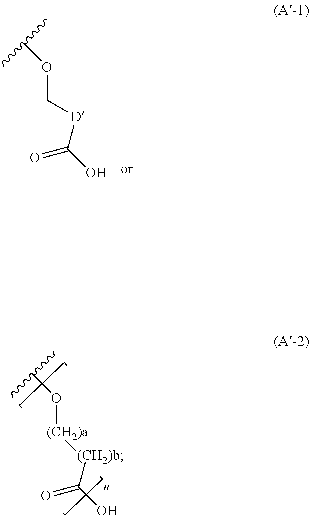
wherein D′ is an optionally substituted C₃ to C₉ aliphatic group that is linear, branched or cyclic, optionally wherein one or more C of the aliphatic group is replaced by —O—, —NH—, —O—C(O)—, or —C(O)—O—;
wherein the aliphatic group optionally comprises at least one substituent —C(O)OH;
wherein n is an integer from 1 to 3; and
wherein each a and b is independently an integer from 1 to 2.

75. An activated biocompatible polymer for use as a medical adhesive, wherein the activated biocompatible polymer is prepared by a process comprising:
obtaining the polymer or the salt or solvate thereof of claim 72, wherein the polymer is prepared by RAFT polymerization;
converting the at least one —C(O)OH group to an activated ester group; and
optionally, isolating the activated biocompatible polymer or a physiologically acceptable salt or solvate thereof.
